(19) United States
(12) Patent Application Publication
(10) Pub. No.: US 2009/0292020 A1
(43) Pub. Date: Nov. 26, 2009

(54) USE OF CALCIUM SALT OF POLYENOIC ACID IN MEDICAL AND COSMETIC FIELD

(76) Inventor: Hui Zhang, Guangdong (CN)

Correspondence Address: DAVID AND RAYMOND PATENT FIRM, 108 N. YNEZ AVE., SUITE 128, MONTEREY PARK, CA, 91754, US

(21) Appl. No.: 12/309,124

(22) PCT Filed: Jun. 18, 2007
(86) PCT No.: PCT/CN2007/001909

§ 371 Date: Jan. 6, 2009

(30) Foreign Application Priority Data
Jul. 7, 2006 (CN) 200610061543.X
Jul. 10, 2006 (CN) 200610061560.3
Jul. 10, 2006 (CN) 200610061561.8
Jul. 26, 2006 (CN) 200610061801.4
Dec. 21, 2006 (CN) 200610157836.8
Dec. 21, 2006 (CN) 200610157837.2
Jan. 12, 2007 (CN) 200710072930.8
Jan. 12, 2007 (CN) 200710072931.2

(51) Int. Cl. A61K 31/20; A61P 17/00

(52) U.S. Cl. 514/560

(57) ABSTRACT

The present invention relates to a process for applying calcium polyenoic acid to medical cosmetology, wherein calcium polyenoic acid, calcium octadecadienic acid and calcium calendic acid are made into ointment or oil-type spraying agent for curing burn, scald, sun burn, bedsore, ulcer, cold injury, trauma. The calcium polyenoic acid and auxiliary materials are made into medical aesthetics product not only for resisting crease, anti-acne, removing mottling, exfoliating scrub, reducing pores and scars, sunburn, abrasion, skin injury, cold injury, antifreezing, anti-cracking, anti-older, sunscreen, repairing and regeneration, repairing damaged skin owing to all kinds of reasons, but also for cosmetic whitening, moisturizing, promoting the growth of epithelial cells, hemorrhoids, rhinitis, repairing damaged organization, promoting cell growth, anti-inflammatory, pain relief, mosquito bites, regulating vascular obstruction, thrombosis, wound healing, inflammation, allergic rhinitis, preventing the artery atherosclerosis and thrombosis formation. The present invention is low cost, high safety and has no side effects for popularization.

### BACKGROUND OF THE PRESENT INVENTION

1. Field of Invention

The present invention relates to medical cosmetology, and more particularly to a process for applying calcium polyenoic acid to medical cosmetology.

2. Description of Related Arts

Now, there are a lot of medicines for burns, sunburn, bed sores, ulcers, cold injury and trauma, most of which are herbal preparations. These herbal preparations have some drawbacks, such as a long treatment time and poor effect.

Most of the medicines for resisting crease, anti-acne, removing mottling, exfoliating scrub, reducing pores and scars, sunburn, abrasion, skin injury, cold injury, antifreezing, anti-cracking, anti-older, sunscreen, repairing and regeneration, repairing damaged skin owing to all kinds of reasons, are herbal preparations having some drawbacks, such as a long treatment time and poor effect.

Most of the medicines for cosmetic whitening, moisturizing, promoting the growth of epithelial cells, hemorrhoids, rhinitis, repairing damaged organization, promoting cell growth, anti-inflammatory, pain relief, and mosquito bites are herbal preparations having some drawbacks, such as a long treatment time and poor effect.

### SUMMARY OF THE PRESENT INVENTION

An object of the present invention is to provide processes of applying calcium polyenoic acid to medical cosmetology.

Process 1 is to prepare calcium polyenoic acid to ointment or oil-type spraying agent, wherein the calcium polyenoic acid is obtained by making polyenoic acid react firstly with NaHCO₃ and then with Ca(OH)₂ solution, or by making polyenoic acid glyceride react with Ca(OH)₂.

A process for obtaining calcium polyenoic acid by polyenoic acid and NaHCO₃, comprises: mixing NaHCO₃ with polyenoic acid to react; stirring; adding Ca(OH)₂ solution after complete reaction; stirring to produce milky calcium polyenoic acid which floating on water surface; layering; and filtrating.

A process for forming calcium polyenoic acid by polyenoic acid glyceride and Ca(OH)₂ solution, comprises: mixing polyenoic acid glyceride with Ca(OH)₂ solution; stirring at normal temperature to produce milky calcium polyenoic acid which floating on water surface; layering; and filtrating.

Prepare calcium polyenoic acid to ointment or oil-type spraying agent. While using, apply the present invention on the disease sites.

The present invention has evident clinic efficacy not only on burn, scald, sun burn, bedsore, ulcer, cold injury, trauma, unhealed cut after surgery, unhealed ulcer lasting a long time caused by inflammation of anal fissure, anal fistula and external genital, but also on beriberi, mosquito bites, and whelk in the inflammatory phase. The present invention is low cost, high safety and has no side effects for popularization.

Process 2 is to prepare calcium octadecadienic acid to ointment or oil-type spraying agent. Dienoic acid is a fatty acid having fatty acid carbon chain with two double-strands. Linoleic acid is the most representative octadecadienic acid in the nature. The calcium octadecadienic acid is obtained by making Linoleic acid react firstly with NaHCO₃ and then with Ca(OH)₂ solution, or by making Linoleic acid glyceride react with Ca(OH)₂.

A process for obtaining calcium octadecadienic acid by NaHCO₃ and linoleic acid, comprises: mixing NaHCO₃ with linoleic acid to react; stirring; adding Ca(OH)₂ solution after complete reaction; stirring to produce milky calcium linoleic acid which floating on water surface; layering; and filtrating. The corresponding chemical reaction equations are shown as:

C₁₈H₃₂O₂+NₐHCO₃═Nₐ[C₁₈H₃₁O₂]+CO₂+H₂O

2Nₐ[C₁₈H₃₁O₂]+Cₐ(OH)₂═Cₐ[(C₁₈H₃₁O₂)]₂+2NₐOH

A process for obtaining calcium octadecadienic acid by linoleic acid glyceride and Ca(OH)₂, comprises: mixing linoleic acid glyceride with Ca(OH)₂ solution; stirring at normal temperature to produce milky calcium linoleic acid which floating on water surface; layering; and filtrating. The corresponding chemical reaction equation is shown as:

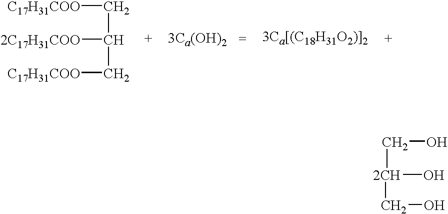

Owing to the different arrangement of molecular structure, octadecadienic acid has several kinds of geometrical isomers which have no influence on efficacy.

Prepare calcium octadecadienic acid to ointment or oil-type spraying agent. While using, apply the present invention on the disease sites.

The present invention has evident clinic efficacy not only on burn, scald, sun burn, bedsore, ulcer, cold injury, trauma, unhealed cut after surgery, unhealed ulcer lasting a long time caused by inflammation of anal fissure, anal fistula and external genital, but also on beriberi, mosquito bites, and whelk in the inflammatory phase. The present invention is low cost, high safety and has no side effects for popularization.

Process 3 is to prepare calcium calendic acid ointment or oil-type spraying agent. trienic acid is a fatty acid having fatty acid carbon chain with three double-strands. Elaeomargaric acid is the most representative linolenic acid in the nature. The calcium calendic acid is obtained by making Elaeomargaric acid firstly react with NaHCO₃ and then with Ca(OH)₂ solution, or by making eleostearic acid glyceride react with Ca(OH)₂.

A process for obtaining calcium calendic acid by NaHCO₃ and elaeomargaric acid comprises: mixing NaHCO₃ with elaeomargaric acid to reacting; stirring; adding Ca(OH)₂ solution after complete reaction; stirring to produce milky calcium eleostearic acid which floats on water surface; layering; and filtrating. The corresponding chemical reaction equations are shown as:

C₁₈H₃₀O₂+NₐHCO₃═Nₐ[C₁₈H₂₉O₂]+CO₂+H₂O

2Nₐ[C₁₈H₂₉O₂]+Cₐ(OH)₂═Cₐ[(C₁₈H₂₉O₂)]₂+2NₐOH

A process for forming calcium calendic acid by eleostearic acid glyceride and Ca(OH)₂ comprising: mixing eleostearic acid glyceride with Ca(OH)₂ solution; stirring at normal temperature to produce milky calcium eleostearic acid which floats on water surface; layering and filtrating. The corresponding chemical reaction equation is shown as:

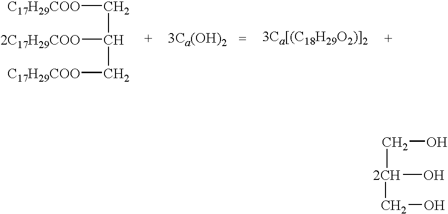

Owing to different arrangement of molecular structure, linolenic acid has several kinds of geometrical isomers which have no influence on efficacy.

Prepare calcium calendic acid to ointment or oil-type spraying agent. While using, apply the present invention on the disease sites.

The present invention has evident clinic efficacy not only on burn, scald, sun burn, bedsore, ulcer, cold injury, trauma, unhealed cut after surgery, unhealed ulcer lasting a long time caused by inflammation of anal fissure, anal fistula and external genital, but also on beriberi, mosquito bites, and whelk in the inflammatory phase. The present invention is low cost, high safety and has no side effects for popularization.

Process 4 is to prepare calcium polyenoic acid to ointment or oil-type spraying agent, wherein the calcium polyenoic acid is obtained by making polyenoic acid react firstly with NaHCO₃ and then with Ca(OH)₂ solution, or by making polyenoic acid glyceride react with Ca(OH)₂.

A process for obtaining calcium polyenoic acid by polyenoic acid and NaHCO₃, comprises: mixing NaHCO₃ with polyenoic acid to react; stirring; adding Ca(OH)₂ solution after complete reaction; stirring to produce milky calcium polyenoic acid which floating on water surface; layering; and filtrating.

A process for forming calcium polyenoic acid by polyenoic acid glyceride and Ca(OH)₂ solution, comprises: mixing polyenoic acid glyceride with Ca(OH)₂ solution; stirring at normal temperature to produce milky calcium polyenoic acid which floating on water surface; layering; and filtrating.

Prepare calcium polyenoic acid to ointment or oil-type spraying agent. While using, apply the present invention on the disease sites.

The present invention has evident clinic efficacy not only on burn, scald, sun burn, bedsore, ulcer, cold injury, trauma, unhealed cut after surgery, unhealed ulcer lasting a long time caused by inflammation of anal fissure, anal fistula and external genital, but also on beriberi, mosquito bites, and whelk in the inflammatory phase. The present invention is low cost, high safety and has no side effects for popularization.

Process 5: the present invention comprises calcium polyenoic acid, glycerine, octadecanol, glycerin monostearate, white mineral oil, white vaselin, P20 emulsifier, propylene glycol, preserving agent K400, essence and water. A compositional ratio of calcium polyenoic acid, glycerine, octadecanol, glycerin monostearate, white mineral oil, white vaselin, P20 emulsifier, propylene glycol, preserving agent K400, essence and water is equal to 15:11:2.5:1:15:7:6:2:0.1:0.1:40.3.

A corresponding process comprises:

1. mixing octadecanol, glycerin monostearate, white mineral oil, white vaselin, P20 emulsifier and heating up the mixture to 95° C.;

2. heating up water to 95° C.;

3. pumping the mixture of step 1 and the water of step 2 simultaneously to a vacuum reactor, heating the mixture up to 85° C. while stirring the mixture, and then cooling the mixture down to 45° C., adding preserving agent K400; adding glycerol 10-propylene glycol and calcium polyenoic acid while cooling down to 42° C., and taking the mixture out from the vacuum reactor after stirring.

The present invention is capable of resisting crease, anti-acne, removing mottling, exfoliating scrub, reducing pores and scars, sunburn, abrasion, skin injury, cold injury, antifreezing, anti-cracking, anti-older, sunscreen, repairing and regeneration, repairing damaged skin owing to all kinds of reasons. The present invention is low cost, high safety and has no side effects for popularization.

Process 6: the present invention comprises calcium polyenoic acid, glycerine, octadecanol, glycerin monostearate, white mineral oil, white vaselin, P20 emulsifier, propylene glycol, preserving agent K400, essence and water. A compositional ratio of calcium polyenoic acid, glycerine, octadecanol, glycerin monostearate, white mineral oil, white vaselin, P20 emulsifier, propylene glycol, preserving agent K400, essence and water is equal to 15:11:2.5:1:15:7:6:2:0.1:0.1:40.3.

Calcium polyenoic acid may be any one of calcium jeceric acid, calcium octadecadienic acid or calcium calendic acid.

A corresponding process comprises:

1. mixing up octadecanol, glycerin monostearate, white mineral oil, white vaselin, P20 emulsifier and heating the mixture up to 95° C.;

2. heating up water to 95° C.;

3. pumping the mixture of step 1 and the water of step 2 simultaneously to a vacuum reactor, heating the mixture up to 85° C. while stirring, and then cooling down to 45° C., adding preserving agent K400; adding glycerol 10-propylene glycol and calcium polyenoic acid while cooling down to 42° C., and taking out the mixture from the vacuum reactor after stirring.

The present invention is capable of resisting crease, anti-acne, removing mottling, exfoliating scrub, reducing pores and scars, sunburn, abrasion, skin injury, cold injury, antifreezing, anti-cracking, anti-older, sunscreen, repairing and regeneration, repairing damaged skin owing to all kinds of reasons. The present invention is low cost, high safety and has no side effects for popularization.

Process 7: the present invention comprises calcium polyenoic acid, glycerine, octadecanol, glycerin monostearate, white mineral oil, white Vaselin, P20 emulsifier, propylene glycol, preserving agent K400, essence and water. A compositional ratio of calcium polyenoic acid, glycerine, octadecanol, glycerin monostearate, white mineral oil, white vaselin, P20 emulsifier, propylene glycol, preserving agent K400, essence and water is equal to 15:11:2.5:1:15:7:6:2:0.1:0.1:40.3.

A corresponding process comprises:

1. mixing up octadecanol, glycerin monostearate, white mineral oil, white vaselin, P20 emulsifier and heating the mixture up to 95° C.;

2. heating up water to 95° C.;

3. pumping the mixture of step 1 and the water of step 2 simultaneously to a vacuum reactor, heating up to 85° C. while stirring, and then cooling down to 45° C., adding preserving agent K400; adding glycerol 10-propylene glycol and calcium polyenoic acid while cooling down to 42° C., taking the mixture out from the vacuum reactor after stirring.

The present invention can be used in cosmetic whitening, moisturizing, promoting the growth of epithelial cells, hemorrhoids, rhinitis, repairing damaged organization, promoting cell growth, anti-inflammatory, pain relief, mosquito bites, regulating vascular obstruction, thrombosis, wound healing, inflammation, allergic rhinitis, preventing the artery atherosclerosis and thrombosis formation. The present invention is low cost, high safety and has no side effects for popularization.

Process 8: the present invention comprises calcium polyenoic acid, glycerine, octadecanol, glycerin monostearate, white mineral oil, white Vaselin, P20 emulsifier, propylene glycol, preserving agent K400, essence and water. A compositional ratio of calcium polyenoic acid, glycerine, octadecanol, glycerin monostearate, white mineral oil, white vaselin, P20 emulsifier, propylene glycol, preserving agent K400, essence and water is equal to 15:11:2.5:1:15:7:6:2:0.1:0.1:40.3.

Calcium polyenoic acid is any one of calcium jeceric acid, calcium octadecadienic acid and calcium calendic acid.

A corresponding process comprises:

1. mixing up octadecanol, glycerin monostearate, white mineral oil, white vaselin, P20 emulsifier and heating the mixture up to 95° C.;

2. heating up water to 95° C.;

3. pumping the mixture of step 1 and the water of step 2 simultaneously to a vacuum reactor, heating the mixture up to 85° C. while stirring, and then cooling down to 45° C., adding preserving agent K400; adding glycerol 10-propylene glycol and calcium polyenoic acid while cooling down to 42° C., and taking the mixture out from the vacuum reactor after stirring.

The present invention can be used in cosmetic whitening, moisturizing, promoting the growth of epithelial cells, hemorrhoids, rhinitis, repairing damaged organization, promoting cell growth, anti-inflammatory, pain relief, mosquito bites, regulating vascular obstruction, thrombosis, wound healing, inflammation, allergic rhinitis, preventing the artery atherosclerosis and thrombosis formation. The present invention is low cost, high safety and has no side effects for popularization.

These and other objectives, features, and advantages of the present invention will become apparent from the following detailed description, and the appended claims.

### DETAILED DESCRIPTION OF THE PREFERRED EMBODIMENT

The present invention is further illustrated by the following embodiment:

### Embodiment 1

Calcium polyenoic acid according to a first preferred embodiment of the present invention is illustrated. Polyenoic acid reacts firstly with NaHCO₃ and then Ca(OH)₂ solution, or polyenoic acid glyceride reacts with Ca(OH)₂ to form the calcium polyenoic acid.

A process for forming calcium polyenoic acid by polyenoic acid and NaHCO₃, comprises: mixing NaHCO₃ with polyenoic acid to react; stirring; adding Ca(OH)₂ solution after complete reaction; stirring and reacting to produce milky calcium polyenoic acid which floats on water surface; layering and filtrating.

A process for forming calcium polyenoic acid by polyenoic acid glyceride and Ca(OH)₂ solution, comprises: mixing polyenoic acid glyceride with Ca(OH)₂ solution; stirring at normal temperature to produce milky calcium polyenoic acid which floats on water surface; layering and filtrating.

The ointment or oil-type spraying agent formed by calcium polyenoic acid of the present invention is capable of smearing on disease sites while using.

### Embodiment 2

Calcium octadecadienic acid according to a second preferred embodiment of the present invention is illustrated. Linoleic acid firstly reacts with NaHCO₃ and then with Ca(OH)₂ solution, or linoleic acid glyceride reacts with Ca(OH)₂ to form calcium octadecadienic acid.

A process for forming calcium octadecadienic acid by NaHCO₃ and linoleic acid, comprises: mixing NaHCO₃ with linoleic acid and reacting; stirring; adding Ca(OH)₂ solution after complete reaction; stirring and reacting to produce milky calcium linoleic acid which floats on water surface; layering; and filtrating. The corresponding chemical reaction equations are shown as:

C₁₈H₃₂O₂+NₐHCO₃═Nₐ[C₁₈H₃₁O₂]+CO₂+H₂O

2Nₐ[C₁₈H₃₁O₂]+Cₐ(OH)₂═Cₐ[(C₁₈H₃₁O₂)]₂+2NₐOH

A process for forming calcium octadecadienic acid by linoleic acid glyceride and Ca(OH)₂, comprises: mixing linoleic acid glyceride with Ca(OH)₂ solution; stirring at normal temperature to produce milky calcium linoleic acid which floats on water surface; layering and filtrating. The corresponding chemical reaction equation is shown as:

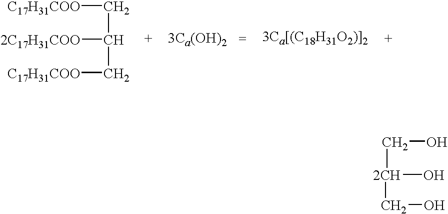

Owing to different arrangement of molecular structure, octadecadienic acid has several kinds of geometrical isomers which have no influence on efficacy.

The ointment or oil-type spraying agent formed by calcium octadecadienic acid of the present invention is capable of smearing on disease sites while using.

### Embodiment 3

Calcium calendic acid according to a third preferred embodiment of the present invention is illustrated. Eleostearic acid firstly reacts with NaHCO₃ and then with Ca(OH)₂ solution, or eleostearic acid glyceride reacts with Ca(OH)₂ to form calcium calendic acid.

A process for forming calcium calendic acid by NaHCO₃ and eleostearic acid comprises: mixing NaHCO₃ with eleostearic acid and reacting; stirring; adding Ca(OH)₂ solution after complete reaction; stirring and reacting to produce milky calcium eleostearic acid which floats on water surface; layering and filtrating. The corresponding chemical reaction equations are shown as:

C₁₈H₃₀O₂+NₐHCO₃═Nₐ[C₁₈H₂₉O₂]+CO₂+H₂O

2Nₐ[C₁₈H₂₉O₂]+Cₐ(OH)₂═Cₐ[(C₁₈H₂₉O₂)]₂+2NₐOH

A process for forming calcium calendic acid by eleostearic acid glyceride and Ca(OH)₂ comprises: mixing eleostearic acid glyceride with Ca(OH)₂ solution; stirring at normal temperature to produce milky calcium eleostearic acid which floats on water surface; layering and filtrating. The corresponding chemical reaction equation is shown as:

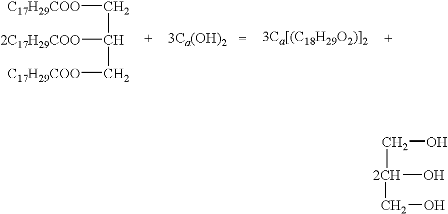

Owing to different arrangement of molecular structure, linolenic acid has several kinds of geometrical isomers which have no influence on efficacy.

The ointment or oil-type spraying agent formed by calcium calendic acid of the present invention is capable of smearing on disease sites while using.

### Embodiment 4

Calcium polyenoic acid according to a fourth preferred embodiment of the present invention is illustrated. Polyenoic acid reacts firstly with NaHCO₃ and then Ca(OH)₂ solution, or polyenoic acid glyceride reacts with Ca(OH)₂ to form the calcium polyenoic acid.

A process for forming calcium polyenoic acid by polyenoic acid and NaHCO₃, comprises: mixing NaHCO₃ with polyenoic acid to react; stirring; adding Ca(OH)₂ solution after complete reaction; stirring and reacting to produce milky calcium polyenoic acid which floats on water surface; layering and filtrating.

A process for forming calcium polyenoic acid by polyenoic acid glyceride and Ca(OH)₂ solution, comprises: mixing polyenoic acid glyceride with Ca(OH)₂ solution; stirring at normal temperature to produce milky calcium polyenoic acid which floats on water surface; layering and filtrating.

The ointment or oil-type spraying agent formed by calcium polyenoic acid of the present invention is capable of smearing on disease sites while using.

### Embodiment 5

A process according to a fifth embodiment of the present invention is illustrated. The present invention comprises calcium polyenoic acid, glycerine, octadecanol, glycerin monostearate, white mineral oil, white vaselin, P20 emulsifier, propylene glycol, preserving agent K400, essence and water. A compositional ratio of calcium polyenoic acid, glycerine, octadecanol, glycerin monostearate, white mineral oil, white vaselin, P20 emulsifier, propylene glycol, preserving agent K400, essence and water is equal to 15:11:2.5:1:15:7:6:2:0.1:0.1:40.3.

A corresponding process comprises:

1. mixing up octadecanol, glycerin monostearate, white mineral oil, white vaselin, P20 emulsifier and heating the mixture up to 95° C.;

2. heating up water to 95° C.;

3. pumping the mixture of step 1 and the water of step 2 simultaneously to a vacuum reactor, heating the mixture up to 85° C. while stirring, and then cooling down to 45° C., adding preserving agent K400, adding glycerol 10-propylene glycol and calcium polyenoic acid while cooling down to 42° C., and taking the mixture out from the vacuum reactor after stirring.

### Embodiment 6

A process according to a sixth embodiment of the present invention is illustrated. The present invention comprises calcium polyenoic acid, glycerine, octadecanol, glycerin monostearate, white mineral oil, white vaselin, P20 emulsifier, propylene glycol, preserving agent K400, essence and water. A compositional ratio of calcium polyenoic acid, glycerine, octadecanol, glycerin monostearate, white mineral oil, white vaselin, P20 emulsifier, propylene glycol, preserving agent K400, essence and water is equal to 15:11:2.5:1:15:7:6:2:0.1:0.1:40.3.

calcium polyenoic acid is any one of calcium jeceric acid, calcium octadecadienic acid and calcium calendic acid.

A corresponding process comprises:

1. mixing up octadecanol, glycerin monostearate, white mineral oil, white vaselin, P20 emulsifier and heating the mixture up to 95° C.;

2. heating up water to 95° C.;

3. pumping the mixture of step 1 and the water of step 2 simultaneously to a vacuum reactor, heating up to 85° C. while stirring, and then cooling down to 45° C., adding preserving agent K400, adding glycerol 10-propylene glycol and calcium polyenoic acid while cooling down to 42° C., and taking the mixture out from the vacuum reactor after stirring.

### Embodiment 7

A process according to a seventh embodiment of the present invention is illustrated. The present invention comprises calcium polyenoic acid, glycerine, octadecanol, glycerin monostearate, white mineral oil, white vaselin, P20 emulsifier, propylene glycol, preserving agent K400, essence and water. A compositional ratio of calcium polyenoic acid, glycerine, octadecanol, glycerin monostearate, white mineral oil, white vaselin, P20 emulsifier, propylene glycol, preserving agent K400, essence and water is equal to 15:11:2.5:1:15:7:6:2:0.1:0.1:40.3.

A corresponding process comprises:

1. mixing up octadecanol, glycerin monostearate, white mineral oil, white vaselin, P20 emulsifier and heating the mixture up to 95° C.;

2. heating up water to 95° C.;

3. pumping the mixture of step 1 and the water of step 2 simultaneously to a vacuum reactor, heating the mixture up to 85° C. while stirring, and then cooling down to 45° C., adding preserving agent K400, adding glycerol 10-propylene glycol and calcium polyenoic acid while cooling down to 42° C., and taking the mixture out from the vacuum reactor after stirring.

### Embodiment 8

A process according to an eighth embodiment of the present invention is illustrated. The present invention comprises calcium polyenoic acid, glycerine, octadecanol, glycerin monostearate, white mineral oil, white vaselin, P20 emulsifier, propylene glycol, preserving agent K400, essence and water. A compositional ratio of calcium polyenoic acid, glycerine, octadecanol, glycerin monostearate, white mineral oil, white vaselin, P20 emulsifier, propylene glycol, preserving agent K400, essence and water is equal to 15:11:2.5:1:15:7:6:2:0.1:0.1:40.3.

Calcium polyenoic acid is any one of calcium jeceric acid, calcium octadecadienic acid and calcium calendic acid.

A corresponding process comprises:

1. mixing up octadecanol, glycerin monostearate, white mineral oil, white vaselin, P20 emulsifier and heating the mixture up to 95° C.;

2. heating up water to 95° C.;

3. pumping the mixture of step 1 and the water of step 2 simultaneously to a vacuum reactor, heating the mixture up to 85° C. while stirring, and then cooling down to 45° C., adding preserving agent K400, adding glycerol 10-propylene glycol and calcium polyenoic acid while cooling down to 42° C., and taking the mixture out from the vacuum reactor after stirring.

One skilled in the art will understand that the embodiment of the present invention as shown in the drawings and described above is exemplary only and not intended to be limiting.

It will thus be seen that the objects of the present invention have been fully and effectively accomplished. It embodiments have been shown and described for the purposes of illustrating the functional and structural principles of the present invention and is subject to change without departure from such principles. Therefore, this invention includes all modifications encompassed within the spirit and scope of the following claims.

1. A process for applying calcium polyenoic acid to medical cosmetology, comprising the steps of:
a step selected from the group consisting of making calcium polyenoic acid into ointment or oil-type spraying agent, making calcium octadecadienic acid into ointment or oil-type spraying agent and making calcium calendic acid into ointment or oil-type spraying agent for burn, scald, sun burn, bedsore, ulcer, cold injury, trauma; and
making said calcium polyenoic acid and auxiliary materials into medical aesthetics product not only for resisting crease, anti-acne, removing mottling, exfoliating scrub, reducing pores and scars, sunburn, abrasion, skin injury, cold injury, antifreezing, anti-cracking, anti-older, sunscreen, repairing and regeneration, repairing damaged skin owing to all kinds of reasons, but also for cosmetic whitening, moisturizing, promoting the growth of epithelial cells, hemorrhoids, rhinitis, repairing damaged organization, promoting cell growth, anti-inflammatory, pain relief, mosquito bites, regulating vascular obstruction, thrombosis, wound healing, inflammation, allergic rhinitis, preventing the artery atherosclerosis and thrombosis formation.

2. The process for applying calcium polyenoic acid to medical cosmetology, as recited in claim 1, wherein said step of making calcium polyenoic acid into ointment or oil-type spraying agent comprises: mixing NaHCO₃ with polyenoic acid to react, stirring the mixture; adding Ca(OH)₂ solution after complete reaction, stirring the mixture to produce milky calcium polyenoic acid which floats on water surface; layering and filtrating.

3. The process of applying calcium polyenoic acid to medical cosmetology, as recited in claim 1, wherein said step of making calcium polyenoic acid into ointment or oil-type spraying agent comprises: mixing Ca(OH)₂ solution with polyenoic acid glyceride; stirring at normal temperature to produce milky calcium polyenoic acid which floats on water surface; layering and filtrating.

4. The process of applying calcium polyenoic acid to medical cosmetology, as recited in claim 1, further comprising a step of making calcium octadecadienic acid into ointment or oil-type spraying agent for burn, scald, sun burn, bedsore, ulcer, cold injury, trauma.

5. The process of applying calcium polyenoic acid to medical cosmetology, as recited in claim 4, wherein said step of making calcium octadecadienic acid into ointment or oil-type spraying agent comprises: mixing NaHCO₃ with linoleic acid and reacting, stirring the mixture; adding Ca(OH)₂ solution after complete reaction, stirring to produce milky calcium linoleic acid which floats on water surface; layering and filtrating.

6. The process of applying calcium polyenoic acid to medical cosmetology, as recited in claim 4, wherein said step of making calcium octadecadienic acid into ointment or oil-type spraying agent comprises: mixing linoleic acid glyceride and Ca(OH)₂ solution; stirring at normal temperature to produce milky calcium linoleic acid which floats on water surface; layering and filtrating.

7. (canceled)

8. The process of applying calcium polyenoic acid to medical cosmetology, as recited in claim 1, further comprising a step of making calcium calendic acid into ointment or oil-type spraying agent for burn, scald, sun burn, bedsore, ulcer, cold injury, trauma; wherein said step of making calcium calendic acid into ointment or oil-type spraying agent comprises: mixing NaHCO₃ with eleostearic acid and reacting, stirring the mixture; adding Ca(OH)₂ solution after complete reaction, stirring to produce milky calcium linoleic acid which floats on water surface; layering and filtrating.

9. The process of applying calcium polyenoic acid to medical cosmetology, as recited in claim 1, further comprising a step of making calcium calendic acid into ointment or oil-type spraying agent for burn, scald, sun burn, bedsore, ulcer, cold injury, trauma; wherein said step of making calcium calendic acid into ointment or oil-type spraying agent comprises: mixing eleostearic acid glyceride and Ca(OH)₂ solution; stirring at normal temperature to produce milky calcium eleostearic acid which floats on water surface; layering and filtrating.

10. (canceled)

11. The process of applying calcium polyenoic acid to medical cosmetology, as recited in claim 1, further comprising a step of making calcium polyenoic acid into ointment or oil-type spraying agent for burn, scald, sun burn, bedsore, ulcer, cold injury and trauma; wherein said step of making calcium polyenoic acid into ointment or oil-type spraying agent comprises: mixing NaHCO₃ with polyenoic acid and reacting, stirring the mixture; adding Ca(OH)₂ solution after complete reaction, stirring to produce milky calcium polyenoic acid which floats on water surface; layering and filtrating.

12. The process of applying calcium polyenoic acid to medical cosmetology, as recited in claim 1, further comprising a step of making calcium polyenoic acid into ointment or oil-type spraying agent for burn, scald, sun burn, bedsore, ulcer, cold injury and trauma; wherein said step of making calcium polyenoic acid into ointment or oil-type spraying agent comprises: mixing Ca(OH)₂ solution and polyenoic acid glyceride; stirring at normal temperature to produce milky calcium polyenoic acid which floats on water surface; layering; and filtrating.

13. The process of applying calcium polyenoic acid to medical cosmetology, as recited in claim 1, further comprising a step of making calcium polyenoic acid into ointment or oil-type spraying agent for burn, scald, sun burn, bedsore, ulcer, cold injury and trauma; wherein said calcium polyenoic acid is adapted for unhealed cut after surgery, unhealed ulcer lasting a long time caused by inflammation of anal fissure, anal fistula and external genital, but also on beriberi, mosquito bites, and whelk in the inflammatory phase.

14. (canceled)

15. The process of applying calcium polyenoic acid to medical cosmetology, as recited in claim 1, wherein said auxiliary materials comprises glycerine, octadecanol, glycerin monostearate, white mineral oil, white vaselin, P20 emulsifier, propylene glycol, preserving agent K400, essence and water; wherein said medical aesthetics product comprises calcium polyenoic acid and auxiliary materials for resisting crease, anti-acne, removing mottling, exfoliating scrub, reducing pores and scars, sunburn, abrasion, skin injury, cold injury, antifreezing, anti-cracking, anti-older, sunscreen, repairing and regeneration, repairing damaged skin owing to all kinds of reasons.

16. The process of applying calcium polyenoic acid to medical cosmetology, as recited in claim 1, wherein said auxiliary materials comprises glycerine, octadecanol, glycerin monostearate, white mineral oil, white vaselin, P20 emulsifier, propylene glycol, preserving agent K400, essence and water, wherein a compositional ratio of calcium polyenoic acid, glycerine, octadecanol, glycerin monostearate, white mineral oil, white vaselin, P20 emulsifier, propylene glycol, preserving agent K400, essence and water is equal to 15:11:2.5:1:15:7:6:2:0.1:0.1:40.3; wherein said medical aesthetics product comprises calcium polyenoic acid and auxiliary materials for resisting crease, anti-acne, removing mottling, exfoliating scrub, reducing pores and scars, sunburn, abrasion, skin injury, cold injury, antifreezing, anti-cracking, anti-older, sunscreen, repairing and regeneration, repairing damaged skin owing to all kinds of reasons.

17. The process of applying calcium polyenoic acid to medical cosmetology, as recited in claim 1, wherein said step of making said medical aesthetics product comprises:
A. mixing up octadecanol, glycerin monostearate, white mineral oil, white vaselin, P20 emulsifier and heating the mixture up to 95° C.;
B. heating up water to 95° C.;
C. pumping said mixture of A and said water of B simultaneously to a vacuum reactor, heating the mixture up to 85° C. while stirring, and then cooling down to 45° C., adding preserving agent K400, adding glycerol 10-propylene glycol and calcium polyenoic acid while cooling down to 42° C., and taking the mixture out from said vacuum reactor after stirring;
wherein said medical aesthetics product comprises calcium polyenoic acid and auxiliary materials for resisting crease, anti-acne, removing mottling, exfoliating scrub, reducing pores and scars, sunburn, abrasion, skin injury, cold injury, antifreezing, anti-cracking, anti-older, sunscreen, repairing and regeneration, repairing damaged skin owing to all kinds of reasons.

18-20. (canceled)

21. The process of applying calcium polyenoic acid to medical cosmetology, as recited in claim 1;
wherein said medical aesthetics product comprises calcium polyenoic acid and auxiliary materials for resisting crease, anti-acne, removing mottling, exfoliating scrub, reducing pores and scars, sunburn, abrasion, skin injury, cold injury, antifreezing, anti-cracking, anti-older, sunscreen, repairing and regeneration, repairing damaged skin owing to all kinds of reasons;
wherein said auxiliary materials comprises glycerine, octadecanol, glycerin monostearate, white mineral oil, white vaselin, P20 emulsifier, propylene glycol, preserving agent K400, essence and water;
wherein said auxiliary materials comprises glycerine, octadecanol, glycerin monostearate, white mineral oil, white vaselin, P20 emulsifier, propylene glycol, preserving agent K400, essence and water, wherein a compositional ratio of calcium polyenoic acid, glycerine, octadecanol, glycerin monostearate, white mineral oil, white vaselin, P20 emulsifier, propylene glycol, preserving agent K400, essence and water is equal to 15:11:2.5:1:15:7:6:2:0.1:0.1:40.3;
wherein said calcium polyenoic acid is selected from a group consisting of calcium jeceric acid, calcium octadecadienic acid and calcium calendic acid.

22. The process of applying calcium polyenoic acid to medical cosmetology, as recited in claim 1;
wherein said medical aesthetics product comprises calcium polyenoic acid and auxiliary materials for resisting crease, anti-acne, removing mottling, exfoliating scrub, reducing pores and scars, sunburn, abrasion, skin injury, cold injury, antifreezing, anti-cracking, anti-older, sunscreen, repairing and regeneration, repairing damaged skin owing to all kinds of reasons;
wherein said auxiliary materials comprises glycerine, octadecanol, glycerin monostearate, white mineral oil, white vaselin, P20 emulsifier, propylene glycol, preserving agent K400, essence and water;
wherein said auxiliary materials comprises glycerine, octadecanol, glycerin monostearate, white mineral oil, white vaselin, P20 emulsifier, propylene glycol, preserving agent K400, essence and water, wherein a compositional ratio of calcium polyenoic acid, glycerine, octadecanol, glycerin monostearate, white mineral oil, white vaselin, P20 emulsifier, propylene glycol, preserving agent K400, essence and water is equal to 15:11:2.5:1:15:7:6:2:0.1:0.1:40.3;
wherein said step of making said medical aesthetics product comprises:
A. mixing up octadecanol, glycerin monostearate, white mineral oil, white vaselin, P20 emulsifier and heating the mixture up to 95° C.;
B. heating up water to 95° C.;
C. pumping said mixture of A and said water of B simultaneously to a vacuum reactor, heating up to 85° C. while stirring, and then cooling down to 45° C., adding preserving agent K400; adding glycerol 10-propylene glycol and calcium polyenoic acid while cooling down to 42° C., and taking the mixture out from said vacuum reactor after stirring.

23. (canceled)

24. The process of applying calcium polyenoic acid to medical cosmetology, as recited in claim 1, wherein said medical aesthetics product comprises calcium polyenoic acid and auxiliary materials for cosmetic whitening, moisturizing, promoting the growth of epithelial cells, hemorrhoids, rhinitis, repairing damaged organization, promoting cell growth, anti-inflammatory, pain relief, mosquito bites, regulating vascular obstruction, thrombosis, wound healing, inflammation, allergic rhinitis, preventing the artery atherosclerosis and thrombosis formation;
wherein said auxiliary materials comprises glycerine, octadecanol, glycerin monostearate, white mineral oil, white vaselin, P20 emulsifier, propylene glycol, preserving agent K400, essence and water.

25. The process of applying calcium polyenoic acid to medical cosmetology, as recited in claim 1, wherein said medical aesthetics product comprises calcium polyenoic acid and auxiliary materials for cosmetic whitening, moisturizing, promoting the growth of epithelial cells, hemorrhoids, rhinitis, repairing damaged organization, promoting cell growth, anti-inflammatory, pain relief, mosquito bites, regulating vascular obstruction, thrombosis, wound healing, inflammation, allergic rhinitis, preventing the artery atherosclerosis and thrombosis formation;
wherein said auxiliary materials comprises glycerine, octadecanol, glycerin monostearate, white mineral oil, white vaselin, P20 emulsifier, propylene glycol, preserving agent K400, essence and water, wherein a compositional ratio of calcium polyenoic acid, glycerine, octadecanol, glycerin monostearate, white mineral oil, white vaselin, P20 emulsifier, propylene glycol, preserving agent K400, essence and water is equal to 15:11:2.5:1:15:7:6:2:0.1:0.1:40.3.

26. The process of applying calcium polyenoic acid to medical cosmetology, as recited in claim 1, wherein said medical aesthetics product comprises calcium polyenoic acid and auxiliary materials for cosmetic whitening, moisturizing, promoting the growth of epithelial cells, hemorrhoids, rhinitis, repairing damaged organization, promoting cell growth, anti-inflammatory, pain relief, mosquito bites, regulating vascular obstruction, thrombosis, wound healing, inflammation, allergic rhinitis, preventing the artery atherosclerosis and thrombosis formation;
wherein said step of making said medical aesthetics product comprises:
A. mixing up octadecanol, glycerin monostearate, white mineral oil, white vaselin, P20 emulsifier and heating the mixture up to 95° C.;
B. heating up water to 95° C.;
C. pumping said mixture of A and said water of B simultaneously to a vacuum reactor, heating the mixture up to 85° C. while stirring, and then cooling down to 45° C., adding preserving agent K400, adding glycerol 10-propylene glycol and calcium polyenoic acid while cooling down to 42° C., and taking the mixture out from said vacuum reactor after stirring.

27-29. (canceled)

30. The process of applying calcium polyenoic acid to medical cosmetology, as recited in claim 1,
wherein said auxiliary materials comprises glycerine, octadecanol, glycerin monostearate, white mineral oil, white vaselin, P20 emulsifier, propylene glycol, preserving agent K400, essence and water;
wherein a compositional ratio of calcium polyenoic acid, glycerine, octadecanol, glycerin monostearate, white mineral oil, white vaselin, P20 emulsifier, propylene glycol, preserving agent K400, essence and water is equal to 15:11:2.5:1:15:7:6:2:0.1:0.1:40.3;
wherein said calcium polyenoic acid is selected from a group consisting of calcium jeceric acid, calcium octadecadienic acid and calcium calendic acid.

31. (canceled)
